(19) United States
(12) Patent Application Publication
(10) Pub. No.: US 2023/0285078 A1
(43) Pub. Date: Sep. 14, 2023

(54) SURGICAL LASER FIBERS AND METHOD FOR MAKING SURGICAL LASER FIBERS HAVING AN ATRAUMATIC DISTAL END

(71) Applicant: Laser Peripherals, LLC, Plymouth, MN (US)

(72) Inventors: Jeffrey M. Stein, Waconia, MN (US); Ricky D. Labrador, Albertville, MN (US)

(73) Assignee: Laser Peripherals, LLC, Plymouth, MN (US)

(21) Appl. No.: 18/113,956

(22) Filed: Feb. 24, 2023

Related U.S. Application Data
(60) Provisional application No. 63/318,990, filed on Mar. 11, 2022.

(51) Int. Cl. A61B 18/22

(52) U.S. Cl. CPC A61B 18/22; A61B 2018/2244; A61B 2018/2222

(57) ABSTRACT

A laser fiber apparatus and method for preparing the apparatus is described herein. An apparatus comprising a laser fiber having a proximal end, a distal end, a core, and a cladding. A lens at the distal end of the laser fiber is proximally elongated from bulbous in shape. The core of the laser fiber transmits laser energy from a laser source coupled to the proximal end through the lens. The cladding of the laser fiber also circumferentially coats at least the proximally elongated portion of the lens, and epoxy material circumferentially coats the cladding, a portion of the lens distal the cladding, and defines an aperture at the apex of the lens.

### CROSS-REFERENCE TO RELATED APPLICATION

This application claims the benefit of U.S. Provisional Application Ser. No. 63/318,990 filed on Mar. 11, 2022, which is incorporated herein by reference in its entirety.

### FIELD OF THE INVENTION

The invention relates to surgical laser fibers and methods of making surgical laser fibers that are compatible with a variety of endoscopes. The inventive surgical laser fibers feature a reinforced distal end having an atraumatic contour that allows the distal end and fiber to navigate unimpeded such as through a deflected endoscopic instrument without snagging, scraping, perforating, or otherwise damaging the endoscope, particularly the layers of the endoscope that lie within the working channel.

### BACKGROUND OF THE INVENTION

Electromagnetic energy, for example laser energy (light) or guided radiation, is used in a variety of surgical and therapeutic procedures for clinical applications. Such laser systems are often used for transmitting light for delivery to a target site to be treated by exposure to the energy. In order to transmit the energy from a source to the target site, laser fibers are often used.

One clinical application that utilizes laser fibers to transmit light energy is flexible ureteroscopy (FURS). FURS involves the use of an endoscope, specifically called a ureteroscope, to enable visualization of the urinary tract. The ureteroscope also includes a lithotomy mechanism to capture or break apart urinary stones. During the FURS procedure, the ureteroscope is guided through the urethra and bladder and up the ureter to the point where the stone is located. In FURS, a laser fiber is introduced into the working channel of the ureteroscope. When the target stone is located, the fiber is advanced a few millimeters beyond the end of the working channel into the operator's field of view, the laser can now be activated, and energy may be applied to the surface of the stone. A lens at the distal end of the fiber is placed near or against the stone, and using the applied light energy, the stone is broken into smaller fragments. Once fragmented, the stone fragments may be captured with a basket and withdrawn through the working channel of the scope. If the fragments will not go through the working channel of the scope, the basket holding the stone remains outside the working channel, and everything is removed in one piece, provided the fragment can be accommodated by the ureter.

Different laser fiber designs and lens shapes have been developed for use in FURS. For example, many known laser fibers have a flat lens with 90° edges at the distal end. Use of laser fibers having such sham-edged lenses increases the risk that the laser fiber can cut into an inner surface of a working channel of an ureteroscope as the laser fiber is inserted and transitions through tight bends in the working channel.

Other known laser fibers developed for use in FURS have a substantially spherical shaped lens at the distal end. A laser fiber having a substantially spherical shaped lens is illustrated by Zerfas et al, in U.S. Pat. No. 10,492,864, the disclosure of which is incorporated herein by reference. In some instances, however, the substantially spherical shaped lens breaks from the laser fiber during the procedure. The potential for the substantially spherical shaped lens of known laser fibers to break or damage the working channel of an endoscope is particularly high when the laser fiber is advanced through a portion of the working channel that is intentionally flexed and bent during a ureteroscopy. This is because lateral forces placed on the substantially spherical shaped lens are generally directed and focused to the narrower exposed portion behind the lens where it connects to the core. A broken lens may result in an undesirable delay during the medical procedure, damage to the scope, or injury to the patient. Thus, a need exists for a laser fiber lens that can safely maneuver through a working channel that is bent and flexed and will not break.

### SUMMARY OF THE INVENTION

The present invention teaches a laser fiber apparatus having a bulbous, atraumatic distal end. The shaped distal end protects the endoscope working channel from damage during initial fiber insertion and facilitates navigation through tight bends without breaking. The apparatus's atraumatic distal end is also reinforced with coatings covering portions of the lens, the cladding, and the core of the laser fiber, resulting in a surgical laser fiber that is stronger than prior art laser fibers and not likely to break. Additionally, the inventive apparatus is advantageous in that no coatings or other materials obstruct the aperture of the lens, thereby improving propagation of the laser energy out of the lens.

The following non-limiting embodiments illustrate certain aspects and features of the laser fiber apparatuses described herein.

One embodiment of the inventive apparatus comprises a laser fiber having a proximal end, a distal end, a core, and a cladding is disclosed. The inventive apparatus may have a lens on the distal end of the laser fiber that is proximally elongated from bulbous in shape. The core of the laser fiber may be capable of transmitting laser energy from a laser source coupled to the proximal end through the lens. The cladding of the laser fiber also circumferentially coats at least the proximally elongated portion of the lens. The apparatus also comprises a curable material, for example UV epoxy, circumferentially coating the cladding, a portion of the lens distal the cladding, and defining an aperture at the apex of the lens.

The laser fiber and lens are preferably a unitary material and a diameter of the aperture is about equal to a diameter of the core.

The minor axis of the lens has a diameter preferably between about 150 μm and about 1400 μm, is preferably between about 1.5 times and about 2.5 times the diameter of the core. The bulbous shape of the lens could be any one of ellipsoid, globose, fusiform, pyriform, tear-shaped, or obovoid.

The laser fiber preferably has an outer coating or buffer, a distal end of the buffer preferably being about 3.5 mm to about 4 mm from of the distal end of the laser fiber.

At least one radiopaque marker is preferably proximal the distal end of the buffer, and at least one radiopaque marker is preferably circumferentially covered with the curable material. Alternatively, the radiopaque marking may be placed distal of the end of the buffer and circumferentially over the coating or cladding layer of the fiber, then covered with curable material. In another embodiment, one may choose to place multiple radiopaque markers in various positions, circumferentially around the fiber layers, and covered with curable material. In this embodiment, the radiopaque markers may be used as a measuring reference. In still another alternative embodiment, the radiopaque marker is applied to the fiber, but is not coated with a curable material.

Another embodiment of the inventive apparatus comprises an optical fiber preferably having a proximal end, a distal end, a core, a cladding, and at least one coating. The navigable surgical fiber preferably has a lens on the distal end of the optical fiber that is funnel-shaped that proximally decreases in diameter from a desired shape. The cladding circumferentially covers a first portion of the lens. The coating terminates a distance from the distal end of the optical fiber, and circumferentially covers a length of the cladding. A curable material overlaps the coating, circumferentially covers the cladding, and defines an aperture of the lens.

The desired shape of the lens in the inventive apparatus is preferably one of ellipsoid, globose, fusiform, pyriform, tear-shaped, bulbous, or obovoid. The lens may be flat at the distal-most end. The flat surface of the lens is preferably coterminous with the aperture at the apex of the lens. One skilled in the art will appreciate that the flat surface of the lens may be perpendicular to an axis of the fiber or at an acute angle to the axis of the fiber.

The optical fiber preferably comprises a coating having at least one radiopaque marker, the at least one coating preferably terminates the distance of about 3 mm to about 4 mm from the distal end of the optical fiber.

The present invention also includes a method of making navigable surgical fibers, comprising removing at least one coating from a distal end of a laser fiber in order to expose a cladding and a core of the laser fiber.

The distal end of the laser fiber is heated and is formed into a lens having a bulbous shape. Preferably a plasma arc or laser finishing system is used to heat the optical fiber and forms the lens into the bulbous shape. The heating process preferably expands the core and cladding of the laser fiber at the distal-most end.

After the formed lens is cooled, a symmetrical coating of light absorbing material is applied to an apex of the lens.

A curable material circumferentially coats the lens, the cladding, and at least about 2 mm of the at least one coating layer and is formed into a substantially cylindrical surface extending from the at least one coating layer to at least the minor axis of the lens.

After the curable material is cured, and a laser energy is transmitted through the laser fiber for about 1 second to about 5 seconds to ablate the light absorbing material and a section of the curable material covering the light absorbing material at the apex of the lens to create a clear aperture.

A radiopaque marker may be applied to a distal end of the at least one coating layer before circumferentially coating with curable material. More than one radiopaque marker may be applied to the at least one coating layer. The more than one radiopaque marker may be placed at specific distances to be used as reference points during clinical applications.

A navigable surgical fiber made by the above method.

### BRIEF DESCRIPTION OF TUE DRAWINGS

FIG. 1 shows a cross-sectional diagram of a preferred embodiment of the claimed laser fiber apparatus;

FIG. 2 shows a cross-sectional diagram of a portion of the laser fiber;

FIG. 3 shows a cross-sectional diagram of a portion of the laser fiber where the buffer and coating were stripped from the cladding;

FIG. 4 shows a cross-sectional diagram of an unfinished embodiment of the laser fiber where a shaped lens was formed at the distal end;

FIG. 5 shows a plain side view of an embodiment of the laser fiber apparatus;

FIG. 6 shows a cross-section diagram of an alternative embodiment of the claimed laser fiber apparatus;

FIG. 7 shows a cross-sectional diagram of an unfinished embodiment of the laser fiber where light absorbing material was applied to the apex of the lens;

FIG. 8 shows a cross-sectional diagram of an unfinished embodiment of the laser fiber where a curable material was applied to the distal end of the laser fiber;

FIG. 9 shows a plain side view of an unfinished embodiment of the laser fiber apparatus with radiopaque material;

FIG. 10 shows a plain side view of an embodiment of the laser fiber apparatus with radiopaque material coated in curable material; and

FIG. 11 is a flowchart depicting a manufacturing process of an embodiment of the laser fiber apparatus.

### DETAILED DESCRIPTION

The detailed description set forth below in connection with the drawings is intended as a description of the particular embodiments of the apparatus and method of making the apparatus and is not intended to represent the only forms in which the present invention may be utilized or constructed. It is to be understood, however, that the same or equivalent functions and methods may be accomplished by different embodiments that are also intended to be encompassed within the spirit and scope of the invention. Throughout the specification, wherever practicable, like structures will be identified by like reference numbers.

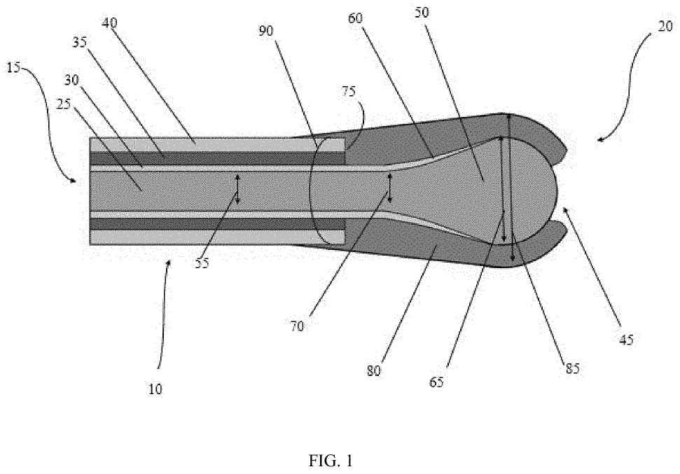

Referring now to FIG. 1, an embodiment of an inventive laser fiber apparatus 10 having a proximal end 15, a distal end 20, a core 25, a cladding 30, an inner coating 35, and an outer coating, or buffer, 40 is shown. The laser fiber apparatus 10 may also be referred to as an optical fiber, navigable surgical fiber, or laser fiber. The core 25 of the laser fiber 10 is generally longitudinally extending and is capable of transmitting laser energy from a light source, coupled to the proximal end 15, through an aperture 45 of the lens 50 at the distal end 20. The core 25 may be made of glass, silica, silicon, silicone, quartz, plastic, polymer or fluoropolymer. The core diameter 55 is generally between about 150 μm and about 1000 μm, and preferably is about 250 μm to about 575 μm with about 270 μm to about 275 μm being a common example.

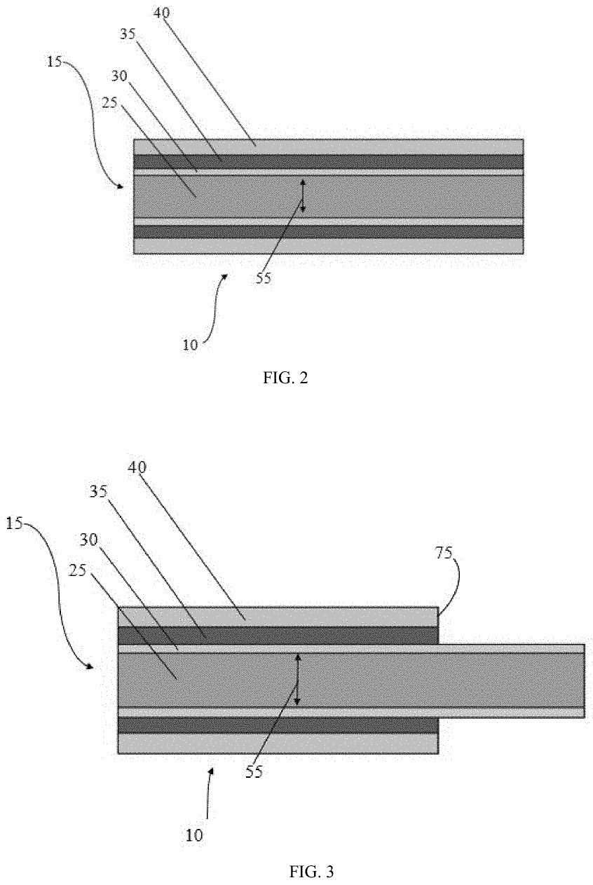

Referring now also to FIG. 2, a portion of the laser fiber apparatus 10 may have at least one cladding layer 30 circumferentially covering the core 25. The cladding layer 30 also circumferentially covers at least the proximally elongated portion 60 of the lens 50. In some embodiments the cladding layer 30 also circumferentially covers a portion of the lens 50 distal the minor axis 65. The cladding preferably has a lower refraction index than the core 25, and may also be made of glass, silica, quartz, plastic, or fluoropolymer. In a preferred embodiment, cladding layer has a diameter of about 295 μm to about 305 inn. The core-to-cladding diameter ratio in this example is around 1:1.1. In some embodiments, multiple cladding layers can be used to circumferentially cover the core 15.

The laser fiber apparatus 10 may also include an inner coating 35 circumferentially covering the cladding layer 30 and core 25. Such inner coating 35 may protect the cladding and the core 25 from scuffs or scratches, additionally adding strength, and may facilitate navigating the laser fiber apparatus 10 through the endoscope by increasing the durability of the apparatus. The coating 35 may be, for example, a fluoropolymer, silicone, or acrylate. One skilled in the art will appreciate the coating 35 could be omitted from the laser fiber 10.

The laser fiber apparatus 10 may also include an outer coating, also referred to as a buffer 40. The buffer 40 may circumferentially cover the inner coating 35. One skilled in the art will appreciate that in some embodiments, the buffer 40 circumferentially covers the cladding 30 because there is no inner coating. In other embodiments, the buffer can act as an additional cladding.

Referring now also to FIG. 3, the coating 35 and the buffer 40 are removed from a portion of the laser fiber apparatus 10. For example, in some embodiments the distal-most end 75 of the coating 35 and buffer 40 is preferably about 3.5 mm to about 4 mm from the distal end 20 of the laser fiber 10. The buffer 40 may be an ethylene tetrafluoroethylene modified copolymer available under the designation “Tefzel” from The Chemours Company of Wilmington, Del. The buffer acts to protect the device from the environment in which it is used. One skilled in the art will appreciate the buffer may also be Hytrel®, nylon, polyimide, silicone, acrylate, or polymers. The outer diameter of the buffer 40 is preferably between 350 μm and 1200 μm, and in a most preferred embodiment the buffer diameter is about 400 μm.

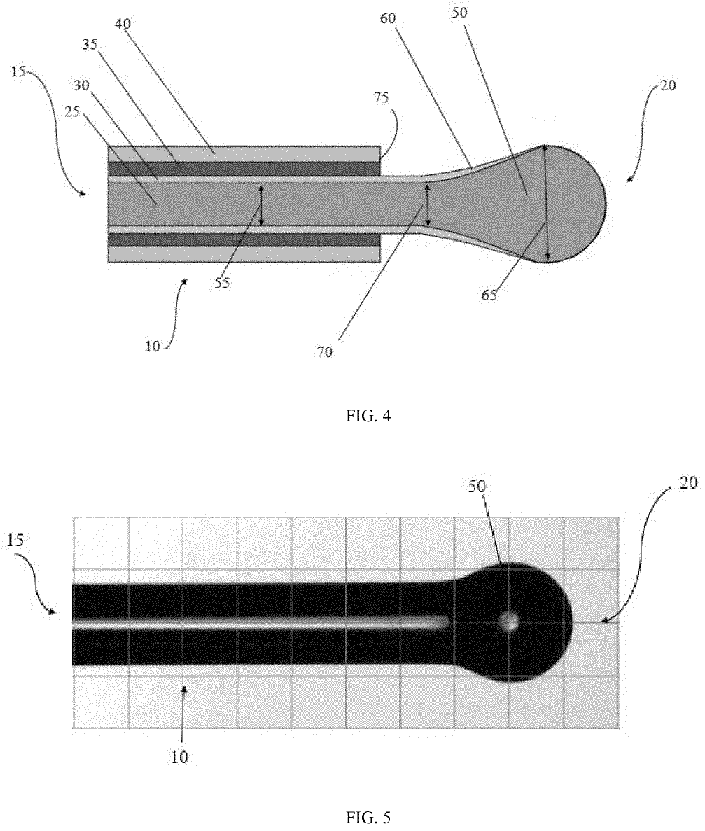

Referring now to FIGS. 4 and 5, at the distal end 20 of the laser fiber 10 is a desired-shaped lens 50 having the advantageous atraumatic contour. The atraumatic contoured lens 50 has a generally proximally elongated portion 60 from a desired shape, for example, bulbous, as shown. The desired-shape lens 50 could alternatively be any one of ellipsoid, globose, fusiform, pyriform, tear-shaped, or obovoid, though other shapes may perform as intended. To produce the preferably proximally elongated portion and desired shape lens, the laser fiber is kept in a generally vertical orientation during the manufacturing process, discussed in more detail below. In some embodiments, the contour of the generally proximally elongated portion 60 may resemble a series of continuous arcs or set of arcs that curve distally outwardly to the desired shape.

In other embodiments, the contour of the generally proximally elongated portion 60 may be funnel-shaped or conical. One skilled in the art will appreciate that the proximally funnel-shaped or proximally conical portion of the lens 50 proximally decreases in diameter from the desired shape to the diameter of the core 25 and cladding 30. This contoured lens 50 mitigates lateral forces being focused to a particular point, junction, or edge, by spreading any forces over the surface of the desired shaped lens 50 and reducing the possibility of the lens 50 breaking away from the core 25 and cladding 30 during a surgical procedure. The diameter of the lens at a minor axis 65 is generally between about 150 μm and about 1400 μm, preferably 200 μm to 1400 μm, and optimally about 500 μm and about 600 μm with about 550 μm being a common diameter. One skilled in the art will appreciate that the diameter of the lens at the minor axis is between about 1.5 times and about 2.5 times a diameter 55 of the core 25 and that a diameter 70 of the proximal-most elongated portion 60 is about equal to the diameter 55 of the core 25. In some embodiments the laser fiber and lens are a unitary material.

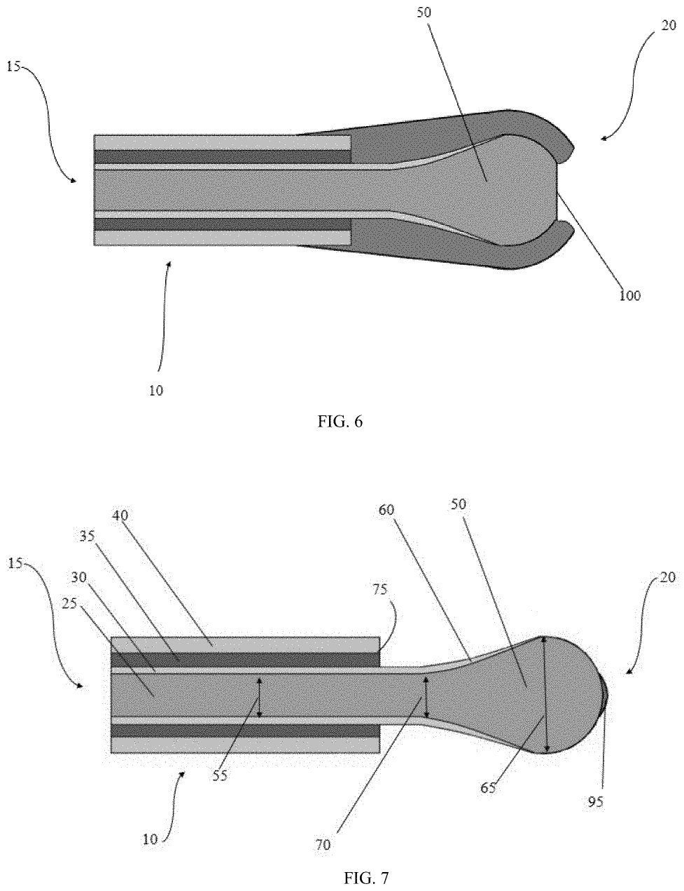

Referring now to FIG. 6, in some embodiments the distal-most portion 20 of the desired shape lens 50 is flat 100. The flat surface 100 may be created by grinding or polishing the desired shape lens, though other methods of creating the flat surface may be used. One skilled in the art will appreciate that the flat surface of the lens may be perpendicular to an axis of the fiber or at an acute angle to the axis of the fiber. Preferably, the flat surface of the lens is coterminous with the aperture at the apex of the lens.

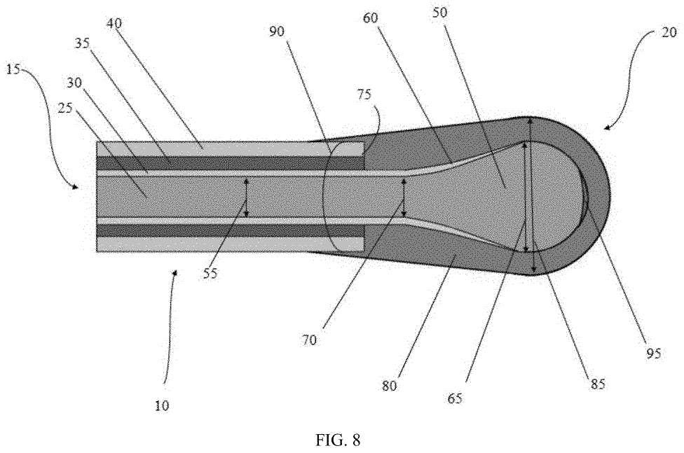

Referring now to FIGS. 7 and 8, a light absorbing material 95 is applied to the apex of the lens at the distal end during the manufacturing process. One skilled in the art will appreciate that in certain embodiments, the light absorbing material 95 is applied to the flattened surface of the lens. The light absorbing material may be, for example, dye or ink, and is preferably a combination of Butanol, Propanol, Diacetone Alcohol, Ethanol, pigments, dyes, and additives. One example of a preferable light absorbing material would be permanent marker, such as a Sharpie® permanent marker. A curable material 80 may overlap the lens 50, the cladding 30, the buffer 40, and the light absorbing material 95. One example of a curable material 80 is UV curable epoxy that includes at least one of an acrylate or silicone. However, one skilled in the art will understand other curable materials may be used. The curable material 80 may also overlap a portion of the buffer 40, for example, the curable material 80 may overlap the buffer 40 by about 2 mm. A UV epoxy—available from MASTERBOND®, 154 Hobard Street, Hackensack, N.J. 07601, branded as UV15 or UV15MED Epoxy—is a suitable Epoxy. The diameter of the UV epoxy coated lens around a minor axis 85 is generally between 215 μm and 1415 μm, and preferably is about 515 μm and about 615 μm with about 565 μm being a common diameter.

After curing the curable material, a portion of the curable material 80 is removed from the lens 50 at the distal-most end of the laser fiber 10. One method of removing the curable material would be chemical removal such as dissolving using Acetone, while another method of removal may be a mechanical means such as physical abrasion or polishing, A preferable method of removing the curable material 80 is to apply a light absorbing material 95 under the curable material 80 during the manufacturing process. Then a laser, for example a holmium laser, is used to ablate the light absorbing material 95 and the curable material 80. The remaining curable material 80 creates an aperture 45 at the apex of the lens 50 that is uncoated, exposed, and transparent. Further, using the preferable method results in more consistent, precise and uniform apertures. One skilled in the art will appreciate other light sources, or methods of removing the curable material 80 may also be employed.

Coating the apex of the lens with the light absorbing material 95 before applying the curable material 80 achieves at least two principal manufacturing advantages. First, the light absorbing material 95 serves as an intermediate coating that inhibits the curable material from adhering directly to the lens 50. Because direct adhesion to the apex of the lens 50 does not occur, complete removal of the curable material 80 from the distal end 20 is substantially facilitated. If, on the other hand, the curable material 80 was not able to be completely removed from the working end of the lens 50, any remaining residual curable material 80 could detrimentally affect propagation of the laser energy to its intended target during use. The intermediate light absorbing material 95, by interfering with the adhesion of the curable material 80 directly to the lens 50, is advantageous in that it facilitates removal of undesirable curable material 80 from the working end of the lens, thereby improving propagation of the laser energy from the lens 50 by creating a clear aperture 45.

Second, the light absorbing material 95 also serves to absorb and retain more heat generated by the laser during ablation than the curable material 80 alone would have (if the curable material were to be adhered directly to the distal end of the lens, rather than to the light absorbing material, prior to ablation). By absorbing and retaining more heat from the laser during ablation, the complete incineration and vaporization of the light absorbing material 95 and the portion of the curable material 80 layer that overlapped the light absorbing material 95 is further facilitated, leaving at aperture 45 at the distal end of the lens 50 clear following ablation. As an example, a holmium laser may be used.

Among other advantages, the curable material 80, upon curing and after ablation, reinforces the strength of the distal portion of the laser fiber 10, particularly at the proximally elongated portion 60 in order to inhibit breakage during use. The curable material 80 also substantially retains the curved, atraumatic contour of the lens 50, that results in a smoother profile for the tip which inhibits scraping or snagging during use.

The standard length of the laser fiber 10 is approximately 3 meters long. Other laser fiber lengths may be implemented as appropriate. The maximum wattage of the laser fiber may be in the range of 1 W and 300 W, preferably 10 W-100 W. In most cases, only the minimum amount of power required for the clinical application is used, as determined by the physicians' training and experience.

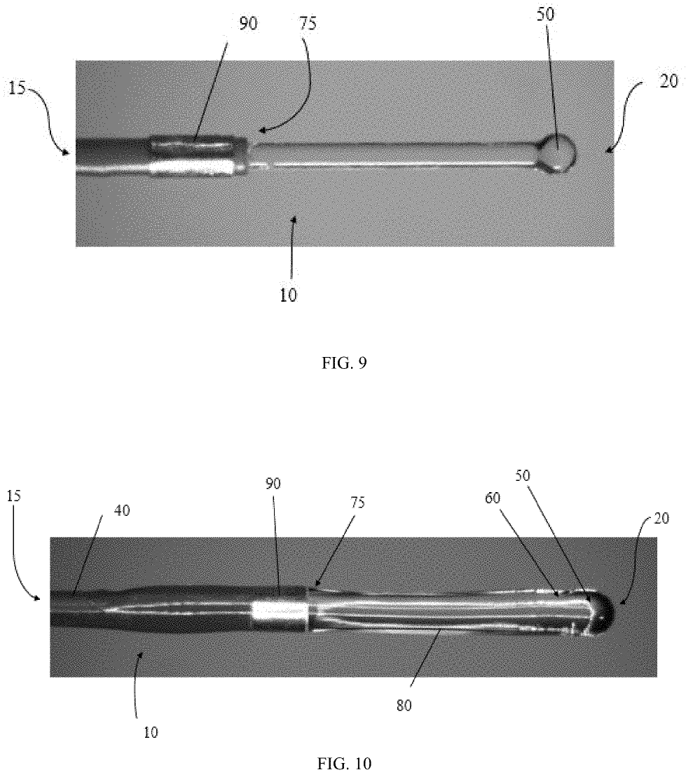

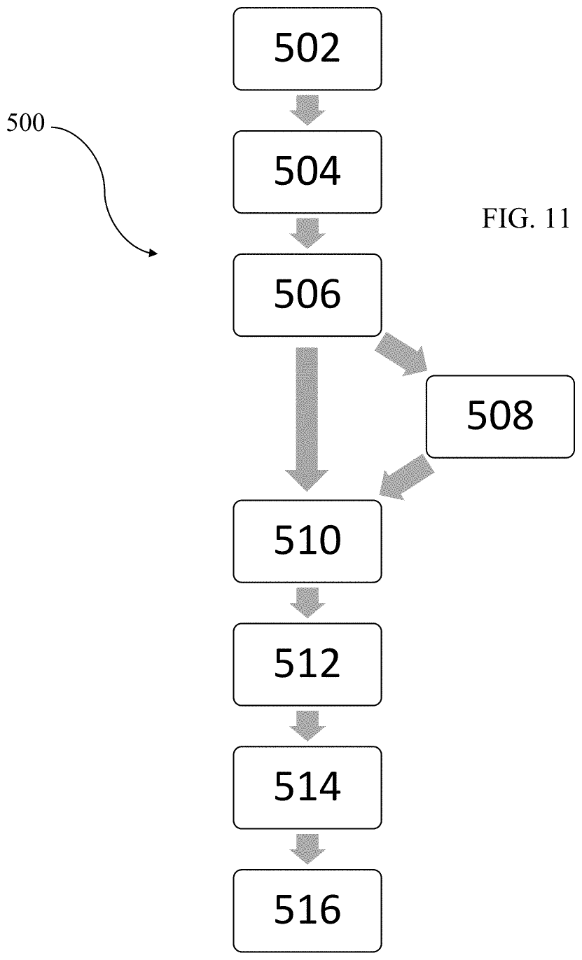

Referring to FIGS. 9 and 10, in various embodiments of the apparatus, radiopaque materials 90 may be used. The radiopaque material 90 may be flush with the distal-most end of the buffer 75. In other embodiments, the radiopaque material 90 may be adjacent the distal-most end of the buffer 75. The radiopaque material 90 may also be spaced along the length of all or part of the laser fiber 10 as dictated by medical procedures facilitated by measured increments from the distal-most end of the laser fiber. The radiopaque material 90 can be any element, such as a foil, wire, or ring that circumferentially covering the buffer 40 or partially covering the buffer 40. Radiopaque foils, wires, rings and any other radiopaque element can be made from gold, platinum, platinum-iridium alloy, tantalum, palladium, silver, bismuth, barium, tungsten, or combinations thereof. The radiopaque material 90 may be a layer, a panel, a reinforcement element, a film or combinations thereof. A plurality of spaced radiopaque materials may also be used. The radiopaque materials may also be etched chemically or with a laser to increase its visibility or imaging artifact.

The laser fiber apparatus 10 may be directly or indirectly attached to a light source. The proximal end 15 of the laser fiber 10 may include a means for attachment to a light source. The means for coupling may include, for example, a stainless-steel ferrule design that mitigates the potential for errant energy to damage the fiber or the laser. In other applications the laser fiber 10 may be used with a connector that has a counter bored tip to allow the proximal end of the fiber to be cantilevered away from the connector components. In yet another application, the proximal end 15 may be circumferentially shrouded using a capillary tubing made of glass, silica, quartz, sapphire, or other optical materials. In this embodiment, the capillary tube is attached to the proximal end 15 using optical epoxy or by fusing the materials together using heat. This method provides further protection by channeling errant energy away from the laser fiber surface to be absorbed by an embedded heatsink within the connector.

FIG. 11 provides a flowchart 500 directed to the preferred method for producing the inventive laser fiber apparatus, an example of which is described below in detail. According to the methods of manufacturing an embodiment of the invention, the laser fiber is processed by a number of steps. These steps are merely exemplary of the order these steps may occur. The steps may occur in any order that is desired such that it still results in the manufacture of an embodiment of the invention

Step 502: The buffer is stripped from approximately the distal-most 3.5 mm to about 4 mm of the distal end of the fiber, leaving the coating exposed at the distal end of the fiber.

Step 504: The stripped distal portion of the fiber is then soaked in acetone for about 1 minute to soften the coating so that it can be physically removed from the approximately 3.5 mm to about 4 mm exposed distal tip. Methanol and a tex wipe may then be used to carefully wipe the exposed distal tip to completely remove any remaining coating, and a microscope may be used to inspect the distal tip to verify that all of the coating has been removed.

Step 506: The stripped fiber is then vertically loaded into the Lens Forming Station “LFS”) to heat the distal tip to melt the exposed glass core and cladding. For example, an LFS—available from 3SAE, 416 Mary Lindsay Polk Drive #511, Franklin, Tenn. 37067, branded as “LFS-01-0100”—is a suitable LFS. The manual of which is incorporated herein by reference. The stripped fiber is fixtured inside and formed within the LFS via a process that takes about 20 seconds to about 30 seconds, and optimally, about 25 seconds at about 1600° Celsius to about 1800° Celsius, and optimally, about 1710° Celsius. The LFS uses a computer-controlled program to measure the loaded fiber, then melt and form the lens by turning on the plasma arc system, then the LFS will stop and measure its progress. The LFS will then repeat the melting, forming, and measuring steps until the desired dimensions and structure are created. These processes in total takes approximately 1 minute from fixturing to removing the fiber from the LFS.

The LFS processor may be used to control the heat application, as well as the vertical position of the distal portion of the core and cladding relative to the heat source, so as to form the desirable shape on the distal end of the laser fiber. During melting, the vertical orientation of the laser fiber and the surface tension forces in the melted core and cladding cause a shaped lens to be formed on the distal tip of the stripped fiber which then cools and solidifies. The melting process causes the shaped lens formed by the melted core and cladding to bulge within the distal end of the cladding layer, which may blend the core and cladding circumferentially over the distal-most portion of the lens after the lens forming process is completed, One skilled in the art will appreciate that different heating times, temperatures, and applications will result in various shaped lenses, including, for example, bulbous, ellipsoid, globose, fusiform, pyriform, tear-shaped, or obovoid, Preferably like shown in FIG. 1, the lens has a bulbous shape. After the lens is formed on the distal end, the lens is cleaned with methanol and a tex wipe to make sure that it is free of contamination.

Step 508: Optionally, the lens is flattened by grinding or polishing the distal-most surface of the lens. Preferably, the flattened surface is coterminous with the diameter of the core. One skilled in the art will appreciate that the flat surface of the lens may be perpendicular or at an acute angle to the axis of the laser fiber.

Step 510: Light absorbing material is applied to the center of the lens at the distal tip, coating the apex of the lens and rendering it substantially opaque. As discussed above, the light absorbing material may be permanent marker or other light absorbing materials. One skilled in the art will appreciate that other light absorbing materials may be used to coat the apex of the lens.

Step 512: The light absorbing material at the apex of the lens, the remaining lens and the distal portion of the fiber is dipped in a reservoir of liquid curable material, for example, UV-curable epoxy, to a depth where the curable material overlaps the buffer of the fiber junction by at least about 2 mm.

Step 514: The curable material coated distal end is rolled using a horizontal motion to ensure a smooth, uniform coating by the curable material. The curable material is then cured using UV light, and the distal portion of the fiber retains the desired shape of the lens. In other embodiments, the distal end may be rolled such that the curable material may have a more funnel or conical shape with a rounded cap. The distal portion of the laser fiber is then inspected under a microscope for uniformity and freedom from contamination.

Step 516: The light absorbing material and curable material are ablated from the apex of the lens. The fiber is coupled, at the proximal end, to a laser source. Preferably a holmium laser is used, however one skilled in the art will appreciate that other lasers can be used for ablation. Laser power of about 5 watts is applied for 3-5 seconds at a setting of 1 joule per pulse, resulting in the light absorbing material and the portion of the curable coating that overlaps the light absorbing material being incinerated at the distal end of the fiber, leaving the distal end of the lens uncoated, exposed, and transparent. The process is controlled by applying a visible, non-destructive laser, such as a HeNe laser, propagated thru the fiber to visibly check that a clear aperture has been formed by imaging a perfectly round exit spot with no light striations.

Preferred embodiments are described herein, including the best mode known to the inventors for carrying out the claimed invention. Variations of those preferred embodiments may become apparent to those of ordinary skill in the art upon reading the foregoing description. Accordingly, the claimed invention includes all modifications and equivalents of the subject matter recited in the claims appended hereto as permitted by applicable law. Moreover, any combination of the above-described elements in all possible variations thereof is encompassed by the claimed invention unless otherwise indicated herein or otherwise clearly contradicted by context.

The embodiments of the invention in which an exclusive property or privilege is claimed are defined as follows:

1. An apparatus comprising:
a laser fiber having a proximal end, a distal end, a core, and a cladding;
a lens on the distal end of the laser fiber and being proximally elongated from bulbous in shape;
the core of the laser fiber being capable of transmitting laser energy from a laser source coupled to the proximal end through the lens;
the cladding of the laser fiber also circumferentially coating at least the proximally elongated portion of the lens; and
an epoxy material circumferentially coating the cladding, a portion of the lens distal the cladding, and defining an aperture at the apex of the lens.

2. The apparatus of claim 1, wherein the laser fiber and lens are a unitary material.

3. The apparatus of claim 1 wherein the bulbous shape is one of ellipsoid, globose, fusiform, pyriform, tear-shaped, or obovoid.

4. The apparatus of claim 1 wherein the diameter of the aperture is about equal to the diameter of the core.

5. The apparatus of claim 1 wherein a minor axis of the lens has a diameter between about 150 μm and about 1400 μm.

6. The apparatus of claim 1 wherein a minor axis of the lens has a diameter between about 1.5 times and about 2.5 times a diameter of the core.

7. The apparatus of claim 1 wherein the epoxy material includes at least one of an acrylate or silicone.

8. The apparatus of claim 1 wherein the laser fiber has a buffer, a distal end of the buffer being about 3.5 mm to about 4 mm from of the distal end of the laser fiber.

9. The apparatus of claim 8 including at least one radiopaque marker proximal the distal end of the buffer.

10. The apparatus of claim 9 wherein at least the distal-most at least one radiopaque marker is circumferentially covered with the epoxy material.

11. A method of making navigable surgical fibers, comprising:
removing at least one coating layer from a distal end of a laser fiber in order to expose a cladding and a core of the laser fiber;
heating the distal end of the laser fiber;
forming the heated distal end of the laser fiber into a lens having bulbous shape;
cooling the lens;
applying a symmetrical coating of light absorbing material to an apex of the lens;
circumferentially coating the lens, the cladding, and a length of the at least one coating layer with curable material;
forming the curable material into a substantially cylindrical surface extending from the at least one coating layer to the minor axis of the lens;
curing the curable material; and
transmitting a laser energy through the laser fiber to ablate the light absorbing material and a section of the curable material covering the light absorbing material at the apex of the lens to create an aperture.

12. A navigable surgical fiber made by the method of claim 11.

13. The method of claim 11 wherein an arc plasma or laser finishing system heats the optical fiber and forms the lens into the bulbous shape.

14. The method of claim 11 wherein a radiopaque marker is applied to a distal end of the at least one coating layer before circumferentially coating with curable material.

15. The method of claim 11 wherein the heating of the core and cladding at the distal-most end of the fiber causes the core and the cladding to melt and bulge which may blend the core and cladding circumferentially over the distal-most portion of the lens.

16. The method of claim 11 wherein more than one radiopaque markers are applied to the at least one coating layer.

17. A navigable surgical fiber comprising:
An optical fiber having a proximal end, a distal end, a core, a cladding, and at least one coating;
a lens on the distal end of the optical fiber, and being funnel-shaped that proximally decreases in diameter from a desired shape;
the cladding circumferentially covering a first portion of the lens;
the at least one coating terminating a distance from the distal end of the optical fiber, and circumferentially covering a length of the cladding;
a curable material overlapping the coating; circumferentially covering the cladding and defining an aperture of the lens.

18. The navigable surgical fiber of claim 17 wherein the desired shape is one of ellipsoid, globose, fusiform, pyriform, tear-shaped, bulbous, or obovoid.

19. The navigable surgical fiber of claim 17 wherein the optical fiber further comprises a coating having at least one radiopaque marker.

20. The navigable surgical fiber of claim 17 wherein the at least one coating terminates the distance of about 3 mm to about 4 mm from the distal end of the optical fiber.
